(19) United States
(12) Patent Application Publication
(10) Pub. No.: US 2024/0360507 A1
(43) Pub. Date: Oct. 31, 2024

(54) METHOD FOR PREDICTING A SEVERITY OF AN INFECTIOUS DISEASE AND BIOMARKER FOR USE IN CARRYING OUT THE METHOD AND MONITORING A THERAPY OF AN INFECTIOUS

(71) Applicant: GENESPECTOR S.R.O., Praha (CZ)

(72) Inventors: Stanislav KMOCH, Praha (CZ); Lenka PIHEROVA, Praha (CZ); Hana HARTMANNOVA, Koberovy (CZ); Michal POHLUDKA, Olomouc (CZ); Martin RADINA, Stary Jicin (CZ)

(21) Appl. No.: 18/684,929

(22) PCT Filed: Dec. 30, 2021
(86) PCT No.: PCT/CZ2021/050159

§ 371 Date: Feb. 20, 2024

(30) Foreign Application Priority Data
Aug. 20, 2021 (CZ) PV 2021-389

(51) Int. Cl. C12Q 1/6876; C12Q 1/6851

(52) U.S. Cl. CPC C12Q 1/6876; C12Q 1/6851; C12Q 2600/106; C12Q 2600/158

(57) ABSTRACT

Method for predicting a severity of an infectious disease and biomarker for use in carrying out the method and monitoring a therapy of an infectious disease using an RT-qPCR method, while the method is being performed on a nasopharyngeal swab sample determining the amount of serum amyloid A mRNA, preferably SAA1, and the amount of serum amyloid A mRNA is normalized to the amount of mRNA of a constitutively expressed gene, which is preferably UBC. Based on the determined normalized value of the amount of SAA1 mRNA in the sample, the severity of the course of an infectious disease, which may be of viral, bacterial, or fungal origin, is predicted, and the effectiveness of a therapy of the given disease is further monitored.

### TECHNICAL FIELD

The invention relates to nucleic acid analysis using molecular biological methods, specifically quantitative polymerase chain reaction (qPCR), testing associated with microorganisms, specifically viruses, bacteria and fungi, and examination of biological materials.

### BACKGROUND ART

Quantitative Polymerase Chain Reaction (qPCR) is a laboratory method used to estimate the copy number of selected nucleic acid segments in the investigated material by monitoring the multiplication efficiency of selected DNA segments, the wide use of which includes, for example, nucleic acid studies, gene analysis and diagnostics, sequencing genetic information or diagnosis of infectious diseases. The principle of PCR consists in the thermal denaturation of the DNA contained in the sample followed by the binding of specific primers to the released DNA strands and the synthesis of new strands with the help of a polymerase enzyme. These steps are repeated in cycles, doubling the amount of DNA in the sample with each run. The Reverse Transcription Quantitative Polymerase Chain Reaction (RT-qPCR) variant allows the determination of the presence of specific RNA, which the reverse transcriptase enzyme present in the reaction mixture transcribes into complementary DNA before amplification itself. In recent years, RT-qPCR has found application, among other things, as a diagnostic method for infectious diseases caused by RNA viruses with excellent high sensitivity and specificity.

During the pandemic of SARS-CoV-2 virus infection and Covid-19 disease, the analytical capacity of state and private laboratories increased significantly and RT-qPCR became a common diagnostic method with non-invasive nasopharyngeal swabs being the primary source of samples. The RT-qPCR method makes it possible to determine very precisely the presence and number of viral RNA molecules (viral load) in the body of the diagnosed patient. This parameter is expressed by the Ct value (Cycle Threshold). However, the Ct value is of diagnostic value only. Due to the specific nature of Covid-19, Ct cannot determine or predict its future clinical course. Low viral load (demonstrated by higher Ct values) can be found in patients with a subsequent severe course of the disease, and conversely, in many cases, high viral load (demonstrated by low Ct values) is found in individuals who do not have or do not develop any clinical symptoms of respiratory disease.

The course of SARS-CoV-2 infection cannot be reliably predicted yet. Also, reliable predictive biomarkers are not known for the transition to a protracted course of the disease (so-called post-COVID syndrome). Significant clinical risk factors leading to a serious course of the disease are older age, some chronic diseases, middle-aged male gender, obesity, diabetes, and hypertension. However, it is very likely that other host factors are also important, especially those that are genetically determined and contribute to the body's innate and acquired immune responses against SARS-CoV-2 infection. An important pathogenetic mechanism for the development of systemic involvement in the case of infectious diseases, and thus Covid-19, is the cytokine storm. Under this condition, there is a gradual uncontrollable release of pro-inflammatory cytokines, activation of acute phase proteins, and abnormal mobilization of the immune system resulting in lung damage, acute liver damage, and kidney failure. Early detection of a developing cytokine storm creates room for its modulation, and thus for the prevention of the development of serious clinical conditions.

Generally, an ideal predictive biomarker would be chosen from molecules that are abundant in the primary diagnostic material, are easily measurable, and manifest concentration changes rapidly and significantly depending on the clinical condition of the subject.

A method for predicting the severity of Covid-19 disease by monitoring the levels of inflammatory markers, specifically C-reactive protein (CRP), serum amyloid A (SAA), procalcitonin (PCT), and interleukin-6 in blood serum is known from Chen et al., Am. J. Transl. Res. 2020, 12 (8), 4569-4575. These so-called acute phase proteins are synthesized almost exclusively in hepatocytes. Their production is stimulated during infections or during inflammatory conditions by a wide range of pro-inflammatory cytokines, especially interleukin-6 (IL-6), interleukin-1β (IL-1β), tumor necrosis factor α (TNF-α), interferon-γ (IFN-γ), Transforming Growth Factor (TGF-β), possibly interleukin-8 (IL-8), and a number of transcription factors (NFκB, C/EBP, YY1, AP-2, SAF and Sp1), which regulate cytokine production. A similar method is also discussed in Pieri M., et al., Int. Immunopharmacol. 2021, 95, 10751. However, the described methods have several shortcomings, especially the need for invasive sampling, which involves tissue disruption, complex analysis of levels of individual indicators, and complicated evaluation of the interrelationships of these values. The methods are therefore unsuitable for mass use in diagnostics.

Ziegler et al., Cell 2021 describes a cytologic analysis of the nasopharyngeal swab of Covid-19-positive patients aimed at a broad complex of substances associated with cytokine and interferon signaling pathways. The possibility of predicting a severe course of the disease is also discussed here, since in contrast to patients with mild and moderate course, in which increased expression of this set of signaling substances was observed, patients with severe course showed low values comparable to the control group of Covid-19-negative individuals. The disadvantage of this method is, as in the previous case, the need to characterize and determine a comprehensive set of indicators with complicated interrelationships and the associated analytical complexity, which makes the method unsuitable for diagnostic use. Furthermore, the non-linear nature of the dependence of the concentration of indicators on the course of the disease does not allow this method to be used to monitor the effectiveness of the applied therapy in the context of disease remission.

The document discussed in the previous paragraph describes, among other things, the determination of SAA in samples from Covid-19-positive patients, where it was observed that in one sample the production of SAA1 and SAA2 is lowered in cells directly affected by SARS-CoV-2 virus and increased in neighboring cells not affected by the virus. This inconsistency and the fact that, according to information available in the expression libraries, the expression of any of the SAA gene family and the associated presence of messenger RNA (mRNA) has not yet been observed in the nasopharyngeal mucosa lead an expert in the art to a conclusion that serum amyloid A it is not a suitable indicator to predict the severity of Covid-19 disease. mRNA SAA has been specifically observed only in breast tissue, gastrointestinal lining, pancreas, prostate, lung, skin, and brain as described for example in Urieli-Shoval S. et al., J. Histochem. Cytochem., 1998, 46(12), 1377-1384.

Outside the bloodstream and physiologically closest to the nasopharynx, the presence of SAA protein has been observed, for example, in porcine saliva (Soler L. et al., Res. Vet. Sci., 2012, 93, 1266-1270). As noted above, SAA protein itself is a widely studied component of the immune response and its levels cannot be correlated with SAA mRNA levels.

Goal of the present invention is to provide a method for predicting the severity of Covid-19 and other infectious diseases in primary diagnostic clinical material-nasopharyngeal swabs-which does not require parallel or subsequent invasive collection of another type of clinical specimen (for example blood or bronchoalveolar lavage), is simple in terms of analytical design, and is based on the determination of one specific indicator, the increase in which can be observed before the onset of symptoms and whose value is directly proportional to the severity of the disease, which further allows monitoring of the course of therapy.

### SUMMARY OF INVENTION

The invention is based on a determination of the amount of serum amyloid A mRNA, preferably SAA1, by the RT-qPCR method in a sample obtained by nasopharyngeal swabs, thus eliminating all the drawbacks of the prior art. Monitoring of SAA1 mRNA levels in nasopharyngeal swabs meets all the criteria of an ideal predictive biomarker. Experiments have shown that SAA1 mRNA is present in the nasopharyngeal swab under physiological conditions and its level is easily measurable by RT-qPCR. SAA1 mRNA levels increase immediately after infection and vary by 3 orders of magnitude (>1000) depending on the extent of inflammation.

SAA1 is an apolipoprotein encoded by the SAA1 gene characterized by the following nucleotide sequence of SEQ ID NO: 1 as follows: AGGCTCAGTATAAATAGCAGCCACCGCTCCCTGGCAGGCAGGGACCCGCAGC TCAGCTACAGCACAGATCAGGTGAGGAGCACACCAAGGAGTGATTTTTAA AACTTACTCTGTTTTCTCTTTCCCAACAAGATTATCATTTCCTTTAAAAAAA ATAGTTATCCTGGGGCATACAGCCATACCATTCTGAAGGTGTCTTATCTCCT CTGATCTAGAGAGCACCATGAAGCTTCTCACGGGCCTGGTTTTCTGCTCCTT GGTCCTGGGTGTCAGCAGCCGAAGCTTCTTTTCGTTCCTTGGCGAGGCTTTT GATGGGGCTCGGGACATGTGGAGAGCCTACTCTGACATGAGAGAAGCCAA TTACATCGGCTCAGACAAATACTTCCATGCTCGGGGGAACTATGATGCTGC CAAAAGGGGACCTGGGGGTGCCTGGGCTGCAGAAGTGATCAGCGATGCCA GAGAGAATATCCAGAGATTCTTTGGCCATGGTGCGGAGGACTCGCTGGCTG ATCAGGCTGCCAATGAATGGGGCAGGAGTGGCAAAGACCCCAATCACTTC CGACCTGCTGGCCTGCCTGAGAAATACTGAGCTTCCTCTTCACTCTGCTCTC AGGAGATCTGGCTGTGAGGCCCTCAGGGCAGGGATACAAAGCGGGGAGAG GGTACACAATGGGTATCTAATAAATACTTAAGAGGTGGAATTTGTGGAAA AAAAAAAAAAAA. Location: (GRCh/hg19) chr11:18287772-18291523. This protein is primarily synthesized in liver and is released into the bloodstream in response to inflammatory stimuli caused by infection, trauma, autoimmune disease, or cancer. SAA1 mRNA has also been found in very small amounts in other tissues, such as adipose tissue, blood vessel wall, intestine, lung, and spleen.

RT-qPCR analysis of nasopharyngeal swabs is a standard that is non-invasive, fast, easy to perform, and easy to automate. This procedure is commonly used not only to diagnose SARS-CoV-2 infection and Covid-19 disease, but also to diagnose other infectious diseases by determining the presence and quantification of virus-specific or microorganism-specific nucleic acids in a sample. As it is possible to test one sample for the presence of more than one target nucleic acid due to the sensitivity of the method, it is possible to obtain not only information on whether an individual is positive for a given viral or other infectious disease, but also to determine SAA1 mRNA parallel, and thus to predict how serious the disease is going to be. Since serum amyloid A is a universal indicator of the inflammatory phase, it is also possible to obtain indications of another serious ongoing infection, which may be of bacterial or fungal origin, in case of a negative result of virological analysis and increased values of SAA1 mRNA.

The mRNA solution purified preferably using magnetic nanoparticles is analyzed by a one-step RT-qPCR reaction in the presence of reverse transcriptase, DNA polymerase, individual primers, and probes in a device that maintains ideal temperature conditions for each step that is cyclically repeated. At the end of each step, the fluorescence of the mixture is always measured, the cycle usually being repeated a total of 40-45 times, and at the end of the program, the Ct values in the individual channels corresponding to the original number of nucleic acid molecules in the nasopharyngeal swab are read.

To quantify the result of SAA1 mRNA analysis and at the same time to avoid skewed results caused by different amounts and sample compositions collected during a nasopharyngeal swab, the amount of mRNA of a selected constitutively expressed gene is determined in parallel and the amount of SAA1 mRNA is related and normalized to it. Constitutively expressed genes are genes that provide basic physiological functions of a cell, and therefore are active and expressed in a constant amount regardless of the state and type of cell. The number of molecules of the selected constitutively expressed gene thus expresses the number of cells present in the harvested material. An example of such a gene is UBC—gene encoding the ubiquitin C protein. Normalization is performed by subtracting the Ct value obtained by RT-qPCR analysis of UBC mRNA in the nasopharyngeal swab sample from the Ct value obtained by RT-qPCR analysis of SAA1 mRNA in the same sample.

According to experimental data, SAA1 mRNA is present in nasopharyngeal swab samples in all patients, including those without ongoing inflammation due to infection, and its level is elevated in the case of ongoing inflammation and is directly proportional to the severity of the course. In the case of severe symptoms requiring hospitalization, the increase in SAA1 mRNA levels is observable even several days before their onset. This also confirms the predictive function of this indicator.

On a statistically significant number of nasopharyngeal swab samples, it was experimentally found out that: 1) normalized SAA1 mRNA levels lower than zero are associated with the absence of ongoing inflammation associated with an infectious disease; and 2) normalized SAA1 mRNA levels higher than zero are associated with an ongoing or developing inflammation associated with an infectious disease, with values lower than one are usually associated with a mild course of inflammation and corresponding disease and values higher than 2.5 are associated with a high probability (95%) of developing inflammation with a serious course with the future need for hospitalization.

The method according to the invention is also suitable for monitoring the effectiveness of a therapy used in the treatment of a given infectious disease. It has been observed that after the application of an appropriate treatment, there is a consequent reduction in the already elevated level of SAA1 mRNA even before the onset of predicted severe symptoms. This method is significantly more accurate than simply monitoring the symptoms, as, for example in Covid-19, symptoms of a respiratory disease such as rhinitis or cough may persist for weeks after the SARS-CoV-2 infection and associated inflammation has been cured.

### BRIEF DESCRIPTION OF DRAWINGS

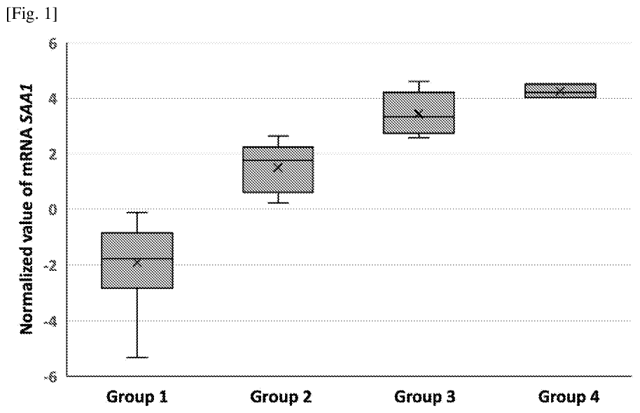

### FIG. 1

FIG. 1 shows a graph of SAA1 mRNA levels normalized to a constitutively expressed UBC gene in patients divided into following groups: Group 1-Healthy individuals and SARS-CoV-2-positive individuals with asymptomatic disease; Group 2-Individuals with mild to moderate course of the infectious disease without the need for hospitalization; Group 3-Hospitalized individuals with severe course of the infectious disease; Group 4-Individuals with a life-threatening course of the infectious disease hospitalized in an intensive care unit. The graph shows the mean (point), median (line), and 95% CI confidence interval (rectangle).

### DESCRIPTION OF EMBODIMENTS

### Example 1

Example 1 describes the nucleotide sequences of primers and probes used to determine the presence of mRNA of SAA1 gene, as well as the sequences of primers and probes used to determine the presence of mRNA of UBC gene (constitutively expressed gene encoding ubiquitin C protein).

Primers and probes used to determine the presence of mRNA of SAA1 gene:

SAA1_upper
(SEQ ID NO: 2)
5′TCGGGGGAACTATGATGCT′3,
Location: (GRCh/hg19) chr11:18290818-18290836
SAA1_lower
(SEQ ID NO: 3)
5′GCACCATGGCCAAAGAATC′3,
Location: (GRCh/hg19) chr11:18291287-18291305
SAA1_probe
(SEQ ID NO: 4)
5′HEX ATCAGCGATGCCAGAGAGAATATCCA BHQ1′3,
Location: (GRCh/hg19) chr11:18291261-18291284
Primers and probes used to determine the presence of mRNA of UBC gene:
UBC_upper
(SEQ ID NO: 5)
5′GATCGCTGTGATCGTCACTTG′3,
Location: (GRCh/hg19) chr12:125399133-125399153
UBC_lower
(SEQ ID NO: 6)
5′GTTTTCCAGCAAAGATCAGCCT′3,
Location: (GRCh/hg19) chr12:125398173-125398194
UBC_probe
(SEQ ID NO: 7)
5′Cy5 TCGTGAAGACTCTGACTGGTAAGACC BHQ2′3,
Location: (GRCh/hg19) chr12:125398282-125398307

### Example 2

Example 2 describes execution of an RT-qPCR assay determining the amount of SAA1 mRNA in a nasopharyngeal swab sample normalized to a parallelly determined amount of mRNA of constitutively expressed UBC gene.

Isolation of mRNA takes place in a solution in the presence of a higher concentration of chaotropic salts, which causes non-covalent binding to hydroxysilane-coated magnetic particles. The mRNA bound to the magnetic particles is washed with solutions containing ethanol or isopropanol and is released into a solution containing no alcohol or chaotropic salts. The obtained mRNA is used directly in a one-step RT-qPCR reaction. This reaction takes place in the presence of MMLV reverse transcriptase, dNTPs, magnesium salts, BSA, hot-start polymerase, 1.6 μM of individual primers, and 0.2 μM of probes listed in Example 1. Reverse transcription takes place for 10 minutes at 50° C., followed immediately by 10 minutes of denaturation at 95° C., in which MMLV reverse transcriptase is inactivated and hot-start polymerase is activated. This is followed by a polymerase chain reaction under the following conditions: denaturation at 95° C. for 10 s and annealing and extension at 58° C. for 30 s. At the end of this step, the fluorescence in the HEX and Cy5 channels is always measured. This cycle is repeated a total of 45 times. At the end of the program, the Ct values in the individual signals are read. Within one sample, the Ct value is obtained in the HEX channel (SAA1) and in the Cy5 channel (UBC). These values correspond to the expression of the individual proteins in the nasopharyngeal swab. The normalized SAA1 mRNA value is obtained by subtracting the UBC mRNA Ct value from the SAA1 mRNA Ct value.

### Example 3

Example 3 demonstrates a realized prediction of severity of the course of an infectious disease in a SARS-CoV-2-positive patient using SAA1 mRNA as a marker.

A sample obtained from a nasopharyngeal swab of an individual showing symptoms of incipient respiratory disease is tested by RT-qPCR for the presence of SARS-CoV-2 viral RNA and for SAA1 mRNA according to the procedure described in Example 2. Ct value obtained for SARS-CoV-2 RNA is 20.46 and the normalized SAA1 mRNA value obtained is 3.48. After seven days, the individual's hospitalization first takes place at the hospital's infectious disease ward and the next day the individual is transferred to the anesthesiology and resuscitation ward.

### Example 4

Example 4 demonstrates a realized confirmation of the efficacy of disease therapy in a SARS-CoV-2-positive patient using SAA1 mRNA as a marker.

A sample obtained from a nasopharyngeal swab of an individual showing symptoms of incipient respiratory disease is tested by RT-qPCR for the presence of SARS-CoV-2 viral RNA and for the presence of SAA1 mRNA according to the procedure described in Example 2. Ct value obtained for SARS-CoV-2 RNA is 21.79 and the normalized SAA1 mRNA value obtained is 2.1. Based on this finding, the subject is subsequently injected with a therapeutic dose of neutralizing antibodies against SARS-CoV-2 virus and, after three days, the RT-qPCR test is repeated for the presence of SARS-CoV-2 viral RNA and SAA1 mRNA according to the procedure described in Example 2. Ct value obtained for SARS-CoV-2 RNA is 29.00 and the normalized SAA1 mRNA value obtained is −2.21, i.e. less than zero. After a further four days, the set of assays is repeated once more, with the SARS-CoV-2 RNA Ct value obtained being 39.64 and the SAA1 mRNA normalized value obtained being −1.5, i.e. less than zero. During a follow-up, the patient does not develop severe symptoms of Covid-19. However, symptoms of respiratory disease in the form of rhinitis and cough persist throughout the follow-up and for several more weeks.

### Example 5

Example 5 demonstrates a realized prediction of severity of the course of an infectious disease of unknown etiology in a SARS-CoV-2-negative patient using SAA1 mRNA as a marker.

A sample obtained from a nasopharyngeal swab of an individual showing symptoms of incipient respiratory disease is tested by RT-qPCR for the presence of SARS-CoV-2 viral RNA and for the presence of SAA1 mRNA according to the procedure described in Example 2. Presence of SARS-CoV-2 RNA is not confirmed and the normalized SAA1 mRNA value obtained is 3.58. Examination of the individual based on clinical symptoms corresponding to an infectious disease in the form of chills, fatigue, and sore throat subsequently reveals a diagnosis of a serious case of bacterial angina.

### Example 6

Example 6 demonstrates a realized prediction of severity of the course of an infectious disease of unknown etiology in a SARS-CoV-2-negative patient using SAA1 mRNA as a marker.

A sample obtained from a nasopharyngeal swab of an individual showing symptoms of incipient respiratory disease is tested by RT-qPCR for the presence of SARS-CoV-2 viral RNA and for the presence of SAA1 mRNA according to the procedure described in Example 2. Presence of SARS-CoV-2 RNA is not confirmed and the normalized SAA1 mRNA value obtained is 4.23. Examination of the individual based on clinical symptoms corresponding to an infectious disease in the form of diarrhea, vomiting, and fever subsequently reveals a diagnosis of a mycosis caused by Candida albicans yeast, i.e. a fungal disease.

### INDUSTRIAL APPLICABILITY

Method for predicting a severity of an infectious disease and biomarker for use in carrying out the method and monitoring a therapy of infectious disease are industrially applicable in diagnostics of infectious diseases based on laboratory analyses of clinical samples.

1. A method for predicting the severity of an infectious disease using RT-qPCR method, comprising:
obtaining a nasopharyngeal swab sample, and
determining the amount of mRNA of serum amyloid A on the nasopharyngeal swab sample.

2. The method according to claim 1, wherein the serum amyloid A is SAA1.

3. The method according to claim 1, wherein the amount of serum amyloid A mRNA is normalized to an amount of mRNA of a constitutively expressed gene.

4. The method according to claim 3, wherein the constitutively expressed gene is UBC.

5. Levels of mRNA of serum amyloid A in nasopharyngeal swab as a biomarker for use in predicting the severity of an infectious disease.

6. Levels of mRNA of serum amyloid A in nasopharyngeal swab as a biomarker for use in monitoring the therapy of an infectious disease.

7. The method according to claim 2, wherein the amount of serum amyloid A mRNA is normalized to an amount of mRNA of a constitutively expressed gene.


SEQUENCE LISTING (7 sequences)

SEQ ID NO: 1 - DNA, 720 bp, Homo Sapiens
aggctcagtataaatagcagccaccgctccctggcaggcagggacccgcagctcagctacagcacagatcaggtgaggagcacaccaaggagtgatttttaaaacttactctgttttctctttcccaacaagattatcatttcctttaaaaaaaatagttatcctggggcatacagccataccattctgaaggtgtcttatctcctctgatctagagagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaaaaaaaa

SEQ ID NO: 2 - DNA, 19 bp, Homo sapiens
tcgggggaactatgatgct

SEQ ID NO: 3 - DNA, 19 bp, Homo sapiens
gcaccatggccaaagaatc

SEQ ID NO: 4 - DNA, 26 bp, Homo sapiens
atcagcgatgccagagagaatatcca

SEQ ID NO: 5 - DNA, 21 bp, Homo sapiens
gatcgctgtgatcgtcacttg

SEQ ID NO: 6 - DNA, 22 bp, Homo sapiens
gttttccagcaaagatcagcct

SEQ ID NO: 7 - DNA, 26 bp, Homo sapiens
tcgtgaagactctgactggtaagacc